(19) United States
(12) Patent Application Publication
(10) Pub. No.: US 2021/0325333 A1
(43) Pub. Date: Oct. 21, 2021

(54) Electrochemical Flow Cell Framework for Evaluating Electroactive Biofilms

(71) Applicant: The Government of the United States of Amerrica, as represented by the Secretary of the Navy, Arlington, VA (US)

(72) Inventors: Sarah M. Glaven, Washington, DC (US); Daniel A. Phillips, Washington, DC (US); Matthew D. Yates, Washington, DC (US); Leonard M. Tender, Bethesda, MD (US)

(21) Appl. No.: 17/233,617

(22) Filed: Apr. 19, 2021

Related U.S. Application Data
(60) Provisional application No. 63/012,437, filed on Apr. 20, 2020.

(51) Int. Cl. G01N 27/327; G01N 27/416; G01N 27/48

(52) U.S. Cl. CPC G01N 27/327; G01N 27/48; G01N 27/4161

(57) ABSTRACT

A biocompatible electrochemical flow cell (eFC) for high resolution imaging of anode and cathode biofilms using laser scanning confocal microscopy employs optically transparent indium tin oxide (ITO)-coated electrode configured to allow observation of the flow chamber. This enables correlation of electrochemical signatures with biofilm development in real-time.

### CROSS-REFERENCE TO RELATED APPLICATIONS

This Application claims the benefit of U.S Provisional Patent Application Ser. No. 63/012,437 filed Apr. 20, 2020, the entirety of which is incorporated herein by reference.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, D.C. 20375, USA; +1.202.767.7230; techtran@nrl.navy.mil, referencing NC 111,917.

### BACKGROUND

Flow cell systems, wherein fresh media is consistently resupplied to a biofilm growing on a solid surface, are regarded as the “gold standard” for studying biofilm morphology and development. Parallel plate flow cells utilize cover glass to allow for visualization of biofilm growth over time. These systems do not easily lend themselves to adaptation for electrochemical measurements, and attempts to retrofit these devices for incorporation of electrodes can compromise the flow cell's structural integrity. When performing microscopy experiments over several days to weeks, leaks within the system are costly, in both time, equipment repair, and reagents.

Flow cells designed with electrochemistry in mind are currently in use. Simultaneous in situ imaging coupled with electrochemical measurements of biofilms during flow cell growth have been reported for anodic bacterial populations (McLean, Wanger et al. 2010, Kitayama, Koga et al. 2017, Du, Mu et al. 2018). In each of these experiments, different flow cells were used to visualize the interaction between the biofilm and the working electrode. Polished graphite working electrodes were frequently used to receive anodic current during biofilm formation (McLean, Wanger et al. 2010, Kitayama, Koga et al. 2017). These designs preclude the usage of transmitted light during the experiment due to the opaque graphite working electrode. A more recent publication used transparent indium tin oxide-coated glass as the working electrode (Du, Mu et al. 2018).

The field of electromicrobiology currently lacks a real-time, adaptable approach to visualize electrode biofilms with microscopy, particularly one suitable for both anodic and cathodic biofilm communities with demonstrated functionality in an aerobic environment. Real-time imaging is critical to advance the field of microbial bioelectronics, where sensors and switches developed to electronically interface bacterial cells with devices must be iteratively tested for temporal response using fluorescence as a benchmark. A need exists for a flow cell operable for facile incorporation of a working, reference, and counter electrode necessary to make basic electrochemical measurements, including chronoamperometry and cyclic voltammetry.

### BRIEF SUMMARY

In one embodiment, an eletrochemical flow cell comprises a flow chamber comprising optically clear material and operably connected to an inlet and an outlet configured to flow growth media through the flow chamber; and three electrodes each exposed to the flow chamber, the electrodes comprising an optically clear indium tin oxide working electrode, a counter-electrode, and a reference electrode, wherein the flow chamber is configured to allow observation of cells therewithin via transmitted illumination that passes through the flow chamber and the indium tin oxide working electrode.

### BRIEF DESCRIPTION OF THE DRAWINGS

FIG. 1 illustrates a single channel electrochemical flow cell (eFC) design.

FIG. 2 provides additional views of the eFC design shown in FIG. 1.

FIG. 3 shows the eFC saddle affixed to the bottom of the eFC. For example, this can be accomplished with double-sided tape after assembly.

FIGS. 4A and 4B show an assembled eFC in use on a confocal microscope and a detailed view of electrode placement after eFC assembly, respectively.

FIG. 5A is an image of Marinobacter atlanticus Strain CP1 pBBR1MCS-2::gfpmut3 in the eFC at 48 hours post-inoculation (“hpi”). FIG. 5B shows measured average current density of multiple experiments with CP1 GFP in the eFC over the course of 48 hours.

FIG. 6A is a full electrode tiling image of CP1 GFP grown in the eFC at 48 hpi. Inset boxes correspond to different locations on the electrode. Arrows indicate direction of media flow. FIG. 6B shows accompanying CV taken during abiotic data collection (black) and at 48 hpi (grey).

FIG. 7 displays chronoamperometry of MCL grown within the eFC over the course of 9 days with continuous flow of fresh, oxygenated artificial sea water (ASW).

FIG. 8 demonstrates how the basic eFC design seen on the left is adaptable to accommodate multiple channels and experimental setups as seen in the middle and right examples.

FIGS. 9A and 9B are show a 3-channel multiplexed eFC design.

### DETAILED DESCRIPTION

Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

Described herein is a biocompatible electrochemical flow cell (eFC) for high resolution imaging of anode and cathode biofilms using laser scanning confocal microscopy. The design employs optically transparent indium tin oxide (ITO)-coated electrodes and enables correlation of electrochemical signatures with biofilm development in real-time. The eFC design can be easily modified to accommodate multiplexed testing of electrochemical reporters, synthetic biology constructs, and conductive living materials in environmentally relevant biofilm formation conditions.

A commercial flow cell retrofitted for electrochemical components is time consuming to set up, spanning across several days, and is also prone to leaking, affecting reproducibility of an experiment and posing a significant risk to sensitive equipment. The described eFC system was designed to accommodate and combine electrochemistry and confocal microscopy with minimal setup time for faster data collection. The fully enclosed, gasket-free nature of the design was developed in order to prevent media leakage, allowing for long-term visualization experiments on fluid-sensitive microscopes. The design is also user friendly and can be mass produced for disposable, single use experiments.

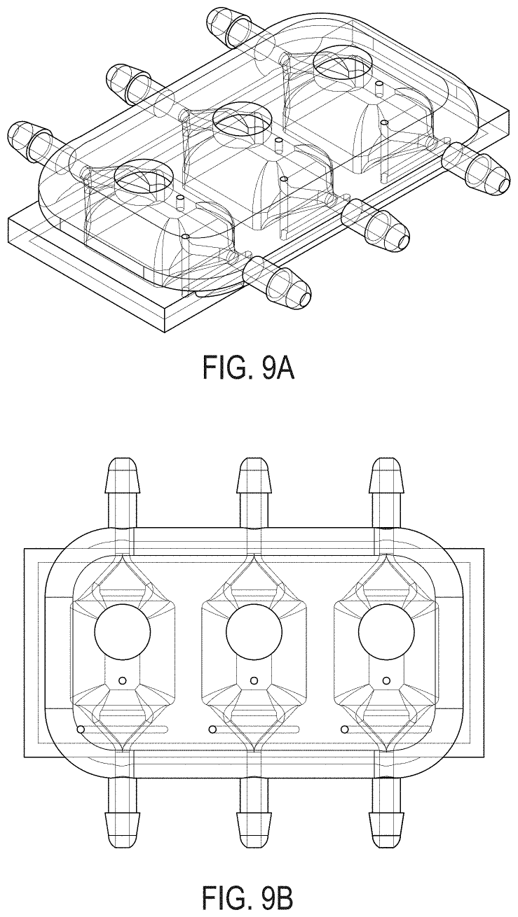

The modularity of the basic flow channel and electrode placement framework allow for rapid, iterative adaptation to novel experimental setups, such as the incorporation of additional flow channels on a single pane of ITO-coated cover glass (FIGS. 8 and 9). This is beneficial over longer experiments at higher resolutions requiring immersion oils. As the microscope objective moves across the surface, the immersion oil can follow the objective. Multiple chambers separated by cover glass gaps or physical barriers would prevent imaging multiple experiments concurrently due to eventual loss of immersion oil. The ability to maintain focus at the electrode surface, record multiple images within a Z-stack series, then move to a different eFC channel overcomes this obstacle, allowing for multiplexed imaging and electrochemical experiments with single cell resolution. Similarly, the design is amenable to incorporation of patterned ITO electrodes, such as an interdigitated microelectrode array (IDA) (Tender, Worley et al. 1996), facilitating expanded electrochemical measurements, including biofilm conductivity.

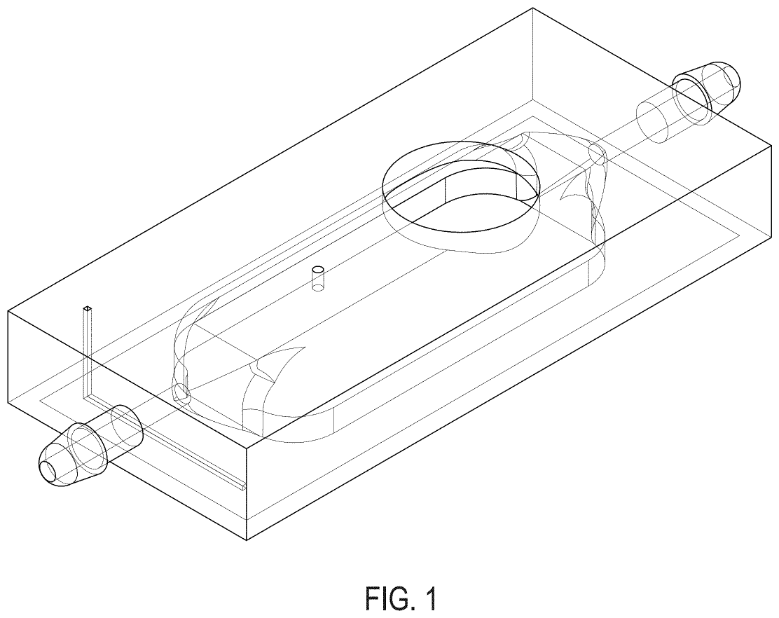

FIG. 1 illustrates an exemplary single channel electrochemical flow cell (eFC) design, having overall dimensions (exclusive of protruding inlet and outlet ports) of about 66 mm length, 30 mm width, and 10 mm height. This allows the eFC to be conveniently visualized on a conventional microscope via transmitted illumination that passes through both the optically clear flow chamber body and an optically clear indium tin oxide electrode.

### EXAMPLES

M. atlanticus CP1 strains were grown on agar plates of “BB” (using one half lysogeny broth and one half Difco Marine Broth) with 100 μg/mL kanamycin at 30° C. as described previously (Bird et al, 2018; Onderko et al, 2019). Briefly, 3 mL cultures of M. atlanticus CP1 strains were grown in low CaCl₂ artificial seawater (1cASW, 0.05 g/L CaCl₂.2H₂O) with 26 mM sodium succinate dibasic hexahydrate as a carbon source and 100 μg/mL kanamycin (Bird et al, 2018; Onderko et al, 2019) overnight to an optical density at 600 nm wavelength (OD₆₀₀) of approximately 0.3. For inoculation into the eFC, 100 μL of this culture was diluted in 6 mL ASW in a 10 mL syringe prior to injection. All Marinobacter atlanticus CP1 strains were grown in the eFC under a constant flow rate of 0.1 mL/min with ASW containing 26 mM succinate and 100 μg/mL kanamycin while kept at 30° C. within the enclosed microscope chamber environment.

Biocathode MCL (designed for its main bacterial components, Marinobacter, Chromatiaceae, and Labrenzia) community cultivation was performed as previously described (Wang et al, 2015). MCL inoculum for the eFC was prepared from a 3 cm×3 cm piece of carbon cloth from the source reactor working electrode. The cloth was shredded into small pieces using an ethanol and flame-sterilized razor blade, and the cloth shreds were placed in 15 mL ASW (Wang, Leary et al. 2015, Eddie, Wang et al. 2016). The inoculum was then vortexed at maximum speed for 30 seconds, sonicated in a sonicating water bath for 20 seconds, and vortexed again for 30 seconds. To inoculate the eFC, 1 mL of the MCL inoculum was aspirated into a syringe containing 9 mL ASW through a 23 G needle before injecting into the inoculation port. The inoculum was incubated without flow for 1 hour to allow for attachment to the ITO surface. After the attachment period, fresh ASW under a continuous flow rate of 0.5 mL/min replaced the medium for the Biocathode MCL biofilm within the eFC.

Confocal images were taken with a Zeiss AxioObserver.Z1/7 LSM 800 Airyscan confocal microscope. Z-stack confocal fluorescence images were recorded at 15 minute intervals over 48 hours with a Plan-Apochromat 40×/1.3 Oil DIC (UV) VIS-IR M27 objective. Full electrode tiling images were taken with an EC Plan-Neofluar 10×/0.3 M27 objective w/0.5×zoom to maximize viewing area per frame. GFPmut3 fluorescence was excited with 488 nm at 0.20% laser power. The emission spectra of GFPmut3 were collected with 464-592 nm filters and detected with the LSM 800 Airyscan detector. The time course GFPmut3 images have a 1.03 μs pixel dwell with 2.53 s per frame. Time course images and videos are presented as maximum intensity projections composed of 28 optical sections over a 20 gm Z-stack interval (0.72 μm/slice) in a 159.73 μm×159.73 μm field of view. Full electrode tiling images are stitched composites of maximum intensity projections over 6 cm² with 5-9 optical sections per field. Images were collected using the Zeiss Zen Blue imaging software (Carl Zeiss, LLC, Thornwood, N.Y., USA).

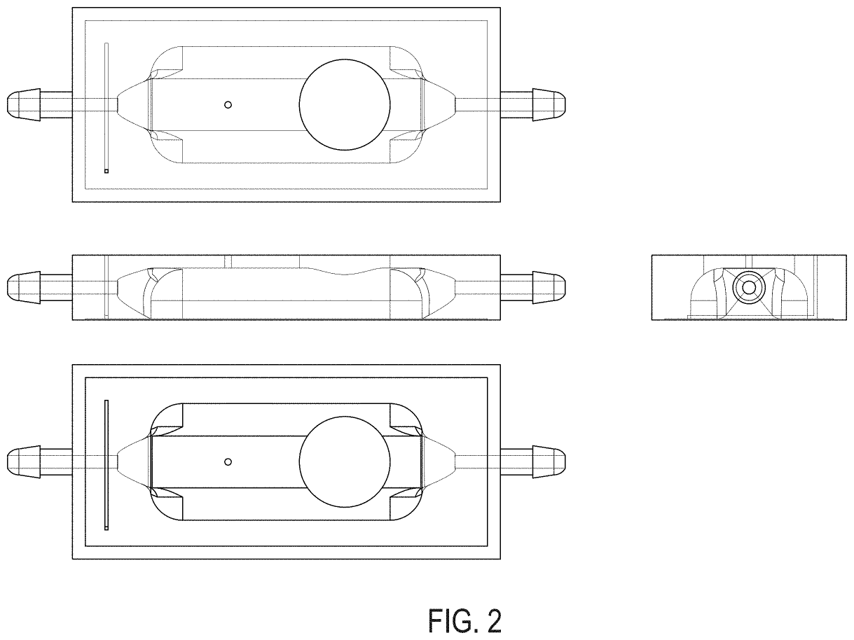

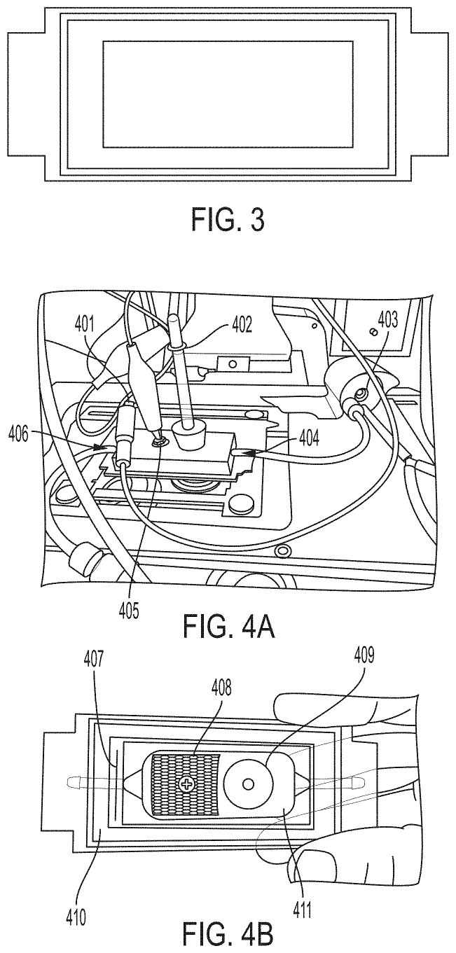

Electrochemical flow cells (eFCs) were designed in Autodesk Inventor 2018 (Autodesk) and printed in biocompatible MED610 resin using a Connex3 Objet500 3D printer (Stratasys) (FIGS. 1 and 2). The eFCs were cleaned with 1% NaOH for 30 minutes after removing the FullCure705 support material. For the counter electrode, 2cm ×2 cm platinum mesh was cleaned with 10% HCl and affixed to the ceiling of the flow chamber with a titanium screw. Transparent indium tin oxide (ITO) coated cover glass (24×60/0.2 mm, Delta Technologies, Ltd, Loveland, Colo.) working electrodes and gold wire contacts were sealed onto the eFC using ARseal™ 90880 polypropylene double-sided adhesive tape (Adhesives Research, Inc.). A second, redundant double-sided tape seal was used to anchor the eFC and ITO cover glass onto the eFC saddle for mounting into the microscope slide holder. An Ag/AgCl₂ reference electrode (3 M KCl, Bioanalytical Systems, Inc.) was slotted through a cored rubber stopper positioned within the flow cell chamber near the working electrode to seal the chamber. Viton tubing (⅛″ ID, 3/16″ OD, 1/32″ wall, Cole-Parmer) segments were linked with Luer lock fittings (Cole-Parmer), and the tubing was slid onto the influx and efflux ports of the eFC. A bubble trap (model 006BT-HF; Omnifit) was positioned immediately upstream of the influx connector tubing, followed by a Y valve for fresh media influx and an inoculation port. Fresh media from the media reservoir bottle is supplied through two-stop PharMed BPT tubing (1.3 mm; ISMATEC) bisecting Viton tubing segments using a peristaltic pump (Ismatec® Reglo; ISMATEC). Tubing extending from the efflux port is terminated with a Luer lock check valve fitting to prevent backflow of media into/through the eFC chamber. Tubing from the check valve extends through a rubber stopper and into the effluent media collection vessel. On the day of each experiment, the eFCs and bubble traps were cleaned with freshly prepared 10% bleach for 5 minutes and rinsed with sterile ddH₂O immediately prior to use. All tubing and glassware are autoclaved to ensure sterility.

For M. atlanticus CP1 anodic growth experiments, working electrodes were poised at 300 mV (vs. Ag/AgCl₂), and chronoamperometry was recorded by a single channel potentiostat (Gamry Instruments Interface 1000) with Gamry Instruments Framework software. Biocathode MCL biofilms were grown on working electrodes poised at 100 mV (vs. Ag/AgCl₂). Non-imaging chronoamperometry replicates of Biocathode MCL biofilms were recorded on a multichannel potentiostat (Solartron 1470E) using the Multistat software (Scribner). Prior to inoculation, abiotic cyclic voltammograms (CVs) were taken, immediately followed by abiotic chronoamperometry measurements until currents stabilized (6-12 h) for all eFC experiments performed. M. atlanticus CP1 chronoamperometry data was collected for 48 hours post inoculation, followed by an endpoint CV under the same conditions as the abiotic CVs.

A transparent indium tin oxide (ITO)-coated cover glass served as the working electrode, facilitating high resolution confocal laser scanning microscopy (CLSM) imaging through the electrode surface and into the bacterial cells in direct contact with the electrode. This technique allows for single cell resolution and direct correlation of electrode biomass to current density. However, a drawback of using ITO as the working electrode for Marinobacter atlanticus strain CP1 biofilms is anodic current production is reported as an order of magnitude less than those grown on graphite electrodes or carbon cloth in 200 mL 3-electrode water-jacketed batch reactors (Onderko, Phillips et al. 2019), likely due to differences in the electrode surface area. This system was designed with electrochemical measurements in mind, rather than retrofitting commercially available flow cell systems with electrode components.

FIGS. 4A and 4B depict an exemplary eFC prepared for use. Electrodes are electrically connected via electrode wire 401 and counter-electrode screw 405. Reference electrode 402 provides voltage reference and is sealed by rubber gasket 409. Fluid is provided through bubble trap 403 and inlet port 404 and efflux moves through outlet port 406. The biocompatible, transparent MED 610 resin 410 provides a structurally sound channel in which bacteria can grow. Transparent, 0.2 mm thick cover glass 411 coated with 7-10 ohm resistance ITO serves as the working electrode on which the biofilm will establish itself within the channel. Gold wire 407 residing in a recessed channel and extending up through the dorsal surface of the eFC serves as the bridge connection between the working electrode and the potentiostat. The non-limiting platinum mesh counter electrode 408 conforms to the curvature of the chamber ceiling and is secured in place with a titanium screw. The titanium screw threads through the eFC body and a rubber washer, providing redundant mechanisms for leak prevention, and is held in place with a titanium nut. The screw 405 provides the contact site for electrical connection to the potentiostat from the counter electrode.

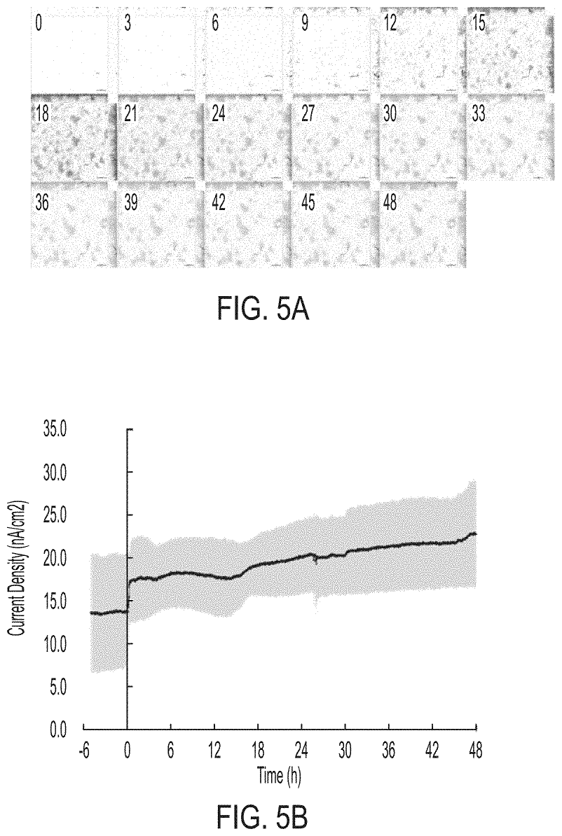

Under continuous flow of fresh artificial seawater (ASW), the ability of Marinobacter atlanticus Strain CP1 to adhere to the ITO electrode in the eFC and produce current densities comparable to those observed in 200 mL, 3 electrode jacketed reactors (Onderko, Phillips et al. 2019) was tested. Cultures from single colonies of M. atlanticus strain CP1 pBBR1MCS-2::gfpmut3 (Bird, Wang et al. 2018) were inoculated by a syringe into the eFC chamber during flow following at least 6 hours of abiotic chronoamperometry data collection. Immediately following inoculation, a field of view was selected based on the presence of few cells attached to the surface to allow for automated focusing and vertical drift control. CLSM z-stack images of CP1 pGFP biofilms were collected simultaneously with chronoamperometry measurements (FIG. 5). Chronoamperometry measurements described here are the average values of three separate experiments. Beginning at around 6 hours post inoculation (hpi), microcolonies of cells begin to appear. Around 12-15 hpi, these colonies develop into vertically growing macrocolonies, and the biofilm takes on a distributed three dimensional architecture. Few cells appear brighter than others, likely due to expression heterogeneity as the biofilm becomes established or localized oxygen availability. At 18 hpi, GFP fluorescence intensity peaks, coincident with a slight but consistent decrease in anodic current. After 18 hpi, anodic current begins increasing again, and GFP fluorescence intensity notably decreases. Previously, anodic current generation from M. atlanticus biofilms was shown to correspond to a decrease in dissolved oxygen tension at this time point in 3 electrode batch reactors (Onderko, Phillips et al. 2019). Likewise, oxygen is necessary for maturation of GFP (Heim, Prasher et al. 1994, Inouye and Tsuji 1994, Heim, Cubitt et al. 1995), so decreased dissolved oxygen tension is a plausible explanation for the apparent decrease in fluorescence intensity during anodic current. As the bacteria grow on the working electrode, metabolism of the succinate in the media results in the generation of hydrogen, CO₂, and electrons that can reduce either the free O₂ in the media or the electrode. Increased biomass, and by extension, CO₂ from metabolism, at the surface of the electrode transitions the growth conditions towards an anoxic environment, making the electrode poised at 300 mV (vs. Ag/AgCl₂) a favorable electron acceptor. By 48 hpi, anodic current plateaus at a current density consistent with values observed in 3 electrode batch reactors.

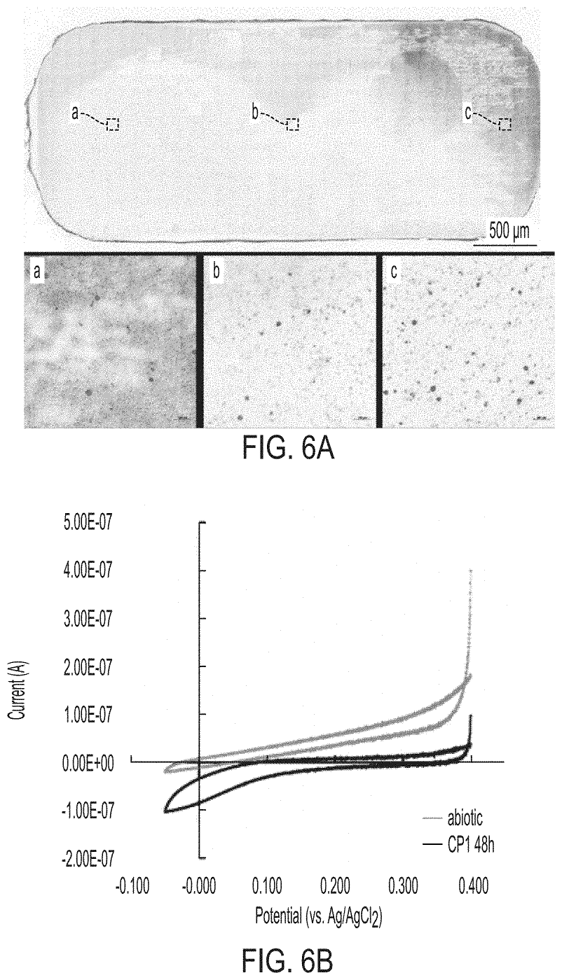

Cyclic voltammograms (CVs) were collected prior to abiotic chronoamperometry and of CP1 pGFP biofilms after 48 hpi (FIGS. 6A and 6B). Representative CVs from a single experiment are shown. Within the abiotic CV, an oxygen reduction peak can be observed at lower potentials. This oxygen reduction peak is not observed in the CP1 pGFP biofilm CV at 48 hpi, supporting the assertion that the CP1 pGFP biomass creates an anoxic zone at the electrode surface. After the final endpoint CV at 48 hpi, whole electrode maximum intensity projection CLSM images were taken and stitched together to visualize biofilm morphology over the entire 6 cm² surface of the electrode (FIG. 6A). The CP1 pGFP biofilm has more biomass and has brighter fluorescence at the inlet of the flow chamber in comparison to the biomass near the interior of the electrode or the outlet of the eFC. Oxygen availability for increased biomass production and GFP maturation is highest at the inlet if the overall dissolved oxygen tension of the ASW decreases as it flows through the eFC chamber. It is noteworthy that a bright ring of GFP fluorescence signal circumscribes the periphery of the eFC chamber. Due to the construction of the eFC, oxygen can become trapped at the interface of the eFC, double-sided tape seal, and the ITO cover glass. Similarly, the biomass growing directly under the position of the reference electrode appears brighter than the rest of the electrode. It is possible that the rubber stopper and reference electrode altered the flow path of the media at this location.

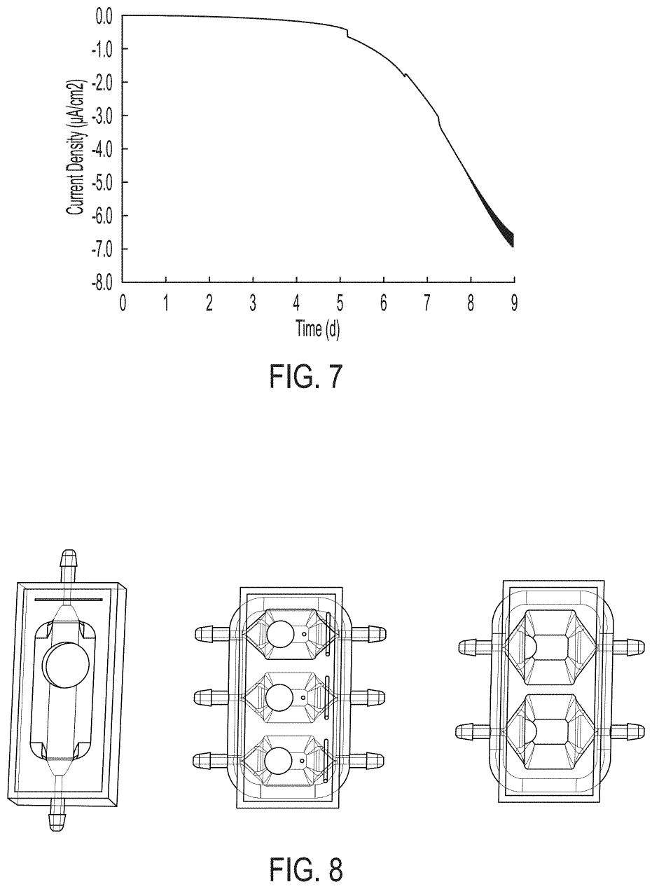

The system is also amenable to growth of electroautotrophic Biocathode MCL biofilms. When grown within the eFC chamber under continuous flow of fresh ASW without an added carbon source, the biofilms produced current densities comparable to those grown in 200 mL 3-chamber batch reactors. After 9 days of growth, current densities reached ˜−7 μA/cm² (FIG. 7), and dense biomass could be observed within the eFC chamber (not shown). The constant supply of oxygenated ASW may play a role in overall biomass accumulation on the electrode.

Further Embodiments

The basic design can be adapted and multiplexed for a variety of eFC configurations. Examples are shown in FIG. 8 of how the system can be multiplexed or adapted to fit the needs of the experiment. Likewise, the system can be used with other working electrode substrates if needed, and could prove useful for alternative imaging methods. For example, electron microscopy grids could be affixed to the eFC working electrode and provide electrochemical, correlative CLSM, and transmission electron microscopy data of the resultant biofilm.

Concluding Remarks

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Bird, L. J., Z. Wang, A. P. Malanoski, E. L. Onderko, B. J. Johnson, M. H. Moore, D. A. Phillips, B. J. Chu, J. F. Doyle, B. J. Eddie and S. M. Glaven (2018). “Development of a Genetic System for Marinobacter atlanticus CP1 (sp. nov.), a Wax Ester Producing Strain Isolated From an Autotrophic Biocathode.” Front Microbiol 9: 3176.

Du, Q., Q. Mu, T. Cheng, N. Li and X. Wang (2018). “Real-Time Imaging Revealed That Exoelectrogens from Wastewater Are Selected at the Center of a Gradient Electric Field.” Environ Sci Technol 52(15): 8939-8946.

Eddie, B. J., Z. Wang, A. P. Malanoski, R. J. Hall, S. D. Oh, C. Heiner, B. Lin and S. M. Strycharz-Glaven (2016). “‘Candidatus Tenderia electrophaga’, an uncultivated electroautotroph from a biocathode enrichment.” Int J Syst Evol Microbiol 66(6): 2178-2185.

Heim, R., A. B. Cubitt and R. Y. Tsien (1995). “Improved green fluorescence.” Nature 373(6516): 663-664.

Heim, R., D. C. Prasher and R. Y. Tsien (1994). “Wavelength mutations and posttranslational autoxidation of green fluorescent protein.” Proc Natl Acad Sci USA 91(26): 12501-12504.

Inouye, S. and F. I. Tsuji (1994). “Evidence for redox forms of the Aequorea green fluorescent protein.” FEBS Lett 351(2): 211-214.

Kitayama, M., R. Koga, T. Kasai, A. Kouzuma and K. Watanabe (2017). “Structures, Compositions, and Activities of Live Shewanella Biofilms Formed on Graphite Electrodes in Electrochemical Flow Cells.” Appl Environ Microbiol 83(17).

McLean, J. S., G. Wanger, Y. A. Gorby, M. Wainstein, J. McQuaid, S. I. Ishii, O. Bretschger, H. Beyenal and K. H. Nealson (2010). “Quantification of Electron Transfer Rates to a Solid Phase Electron Acceptor through the Stages of Biofilm Formation from Single Cells to Multicellular Communities.” Environmental Science & Technology 44(7): 2721-2727.

Onderko, E. L., D. A. Phillips, B. J. Eddie, M. D. Yates, Z. Wang, L. M. Tender and S. M. Glaven (2019). “Electrochemical Characterization of Marinobacter atlanticus Strain CP1 Suggests a Role for Trace Minerals in Electrogenic Activity.” Frontiers in Energy Research 7.

Tender, L. M., R. L. Worley, H. Y. Fan and G. P. Lopez (1996). “Electrochemical patterning of self-assembled monolayers onto microscopic arrays of gold electrodes fabricated by laser ablation.” Langmuir 12(23): 5515-5518.

Wang, Z., D. H. Leary, A. P. Malanoski, R. W. Li, W. J. t. Hervey, B. J. Eddie, G. S. Tender, S. G. Yanosky, G. J. Vora, L. M. Tender, B. Lin and S. M. Strycharz-Glaven (2015). “A previously uncharacterized, nonphotosynthetic member of the Chromatiaceae is the primary CO2-fixing constituent in a self-regenerating biocathode.” Appl Environ Microbiol 81(2): 699-712.

What is claimed is:

1. An electrochemical flow cell comprising:
a flow chamber comprising optically clear material and operably connected to an inlet and an outlet configured to flow growth media through the flow chamber; and
three electrodes each exposed to the flow chamber, the electrodes comprising an optically clear indium tin oxide working electrode, a counter-electrode, and a reference electrode,
wherein the flow chamber is configured to allow observation of cells therewithin via transmitted illumination that passes through the flow chamber and the indium tin oxide working electrode.

2. The electrochemical flow cell of claim 1, wherein the counter-electrode is platinum.

3. The electrochemical flow cell of claim 1, wherein the reference electrode is an Ag/AgCl₂ electrode.

4. The electrochemical flow cell of claim 1, further comprising a potentiostat operably connected to the electrodes.

5. The electrochemical flow cell of claim 1, wherein the clear material is a biocompatible resin.

6. The electrochemical flow cell of claim 1, having dimensions of no greater than about 66 mm×30 mm×10 mm.

7. The electrochemical flow cell of claim 1, wherein said observation of cells is possible without requiring reflection of light within the flow chamber.

8. The electrochemical flow cell of claim 1, wherein the optically clear indium tin oxide working electrode is a cover glass sealed to the flow chamber.

9. An electrochemical flow cell comprising:
a flow chamber comprising optically clear material and operably connected to an inlet and an outlet configured to flow growth media through the flow chamber;
three electrodes each exposed to the flow chamber, the electrodes comprising an optically clear indium tin oxide working electrode, a counter-electrode, and a reference electrode; and
a potentiostat operably connected to at least two of the electrodes,
wherein the flow chamber is configured to allow observation of cells therewithin via transmitted illumination that passes through the flow chamber and the indium tin oxide working electrode.

10. The electrochemical flow cell of claim 9, wherein the optically clear indium tin oxide working electrode is a cover glass sealed to the flow chamber.
